(19) United States
(12) Patent Application Publication
(10) Pub. No.: US 2020/0390367 A1
(43) Pub. Date: Dec. 17, 2020

(54) SYMPTOMATIC TREMOR DETECTION SYSTEM

(71) Applicant: Medic, Inc., Provo, UT (US)

(72) Inventors: David R. Hall, Provo, UT (US); Travis Niederhauser, Mapleton, UT (US); Daniel Hendricks, Provo, UT (US)

(73) Assignee: Medic, Inc., Provo, UT (US)

(21) Appl. No.: 16/899,283

(22) Filed: Jun. 11, 2020

Related U.S. Application Data
(60) Provisional application No. 62/862,418, filed on Jun. 17, 2019.

(51) Int. Cl. A61B 5/11; G16H 10/60; G16H 50/20; G16H 50/30; A61B 5/00

(52) U.S. Cl. CPC A61B 5/1101; G16H 10/60; G16H 50/20; G16H 50/30; A61B 5/1116; A61B 2562/0219; A61B 5/7275; A61B 2505/07; A61B 2560/0468; A61B 2562/0252; A61B 5/6891

(57) ABSTRACT

A symptomatic tremor detection system is disclosed. The system includes a seat sensor to measure at least a portion of weight from a user on a seat and a foot sensor to measure at least a portion of weight from the user on a foot support while the user is on the seat and supporting a foot with a foot support. The data from the seat sensor and the force sensor is used to identify a temporary weight changes in the data which correlate with the manifestation of symptomatic tremors. An additional, or tertiary, force sensor may also be used with the system to provide additional data relevant to the detection of symptomatic tremors.

### CROSS-REFERENCE TO RELATED APPLICATIONS

This application claims priority to U.S. Provisional Patent Application No. 62/862,418 titled “System for Detecting Body Tremors” filed on 17 Jun. 2019, which is incorporated herein by reference in its entirety.

### TECHNICAL FIELD

The present disclosure relates to analytical toilets. More particularly, it relates to analytical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station.” The disclosures of all these patents and applications are incorporated by reference in their entireties.

A body tremor is an involuntary, rhythmic muscle contraction and relaxation leading to shaking movements in one or more parts of the body. These movements may be motion of one part of the body relative to another. The movements may also manifest as vibrations or waves that propagate through the body similar to an earthquake propagating through the Earth from the earthquake origin. Some tremors can be a physical indication or symptom indicating that a person has a condition for which health or other wellness care should be considered. Additionally, changes to the rhythm of a person's typical tremors can also indicate a change in condition for which health or other wellness care should be considered. Some atypical conditions and circumstances that show correlation with abnormal body tremors include multiple sclerosis, stroke, traumatic brain injury, neurodegenerative diseases that affect parts of the brain (such as Parkinson's disease), use of certain medicines (including particular asthma medication, amphetamines, caffeine, corticosteroids, and some drugs used for psychiatric and neurological disorders), alcohol abuse or withdrawal, mercury poisoning, an overactive thyroid, liver or kidney failure, and anxiety or panic.

There are many ways to classify tremors. One method uses two main categories: resting tremor and action tremor. Resting tremors occur when the muscle(s) is relaxed. Action tremors occur with the voluntary use of a muscle, such as standing still, holding something, walking, and purposefully moving an appendage or other part of the body. Some action tremors are specifically related to goal-oriented, skill related tasks, such as writing or speaking. Another way to classify tremors is based on their appearance, cause, and/or origin. Using this method, there are more than 20 types of tremor. Some common classifications used in this methodology include essential, dystonic, cerebellar, psychogenic, physiologic, enhanced physiologic, Parkinsonian, and orthostatic. A third way to classify tremors is by their frequency or how long it takes for the general motion to repeat. Tremor frequency generally falls within the range of 3-30 Hz or 3-30 times per second. Under 4 Hz may be termed low frequency, 4-7 Hz may be termed medium frequency, and over 7 Hz may be termed high frequency. One method of assessing tremor amplitude uses the following displacement categorizations: (a) no tremor, (b) slight tremor, (c) moderate tremor with displacement under 2 cm, (d) marked tremor with displacement between 2 cm and 4 cm, and (e) severe tremor with displacement over 4 cm.

Notably, physiologic tremors occur in all healthy individuals, are generally not associated with a disease, are generally associated with normal human phenomenon such as heartbeat or maintaining a posture or movement, and may manifest as a fine shaking, such as of the hands and fingers, that is rarely visible to the eye. Neurologic examination results of patients with physiologic tremor are usually normal. Physiologic tremors can be exacerbated, making them significantly more noticeable. An exacerbated physiologic tremor may be a cause for concern and additional health or wellness consideration. Exacerbating factors can include extreme fatigue, stress, intense emotion, low blood sugar (hypoglycemia), an overactive thyroid, medications such as corticosteroids, amphetamines or beta-agonists, heavy metal toxicity, stimulants such as caffeine, fever, and alcohol withdrawal.

Diagnosis of a tremor is based on clinical information obtained from a thorough history and physical examination. A possible first step in the evaluation of a patient with a tremor is to categorize the tremor based on the activation condition, topographic distribution, and frequency that correlate with the manifestation of the tremor. Additional steps can follow if necessary.

Some examples of devices that can detect tremors include U.S. Pat. No. 4,595,023 titled “Apparatus and Method for Detecting Body Vibrations”, U.S. Pat. No. 6,936,016 titled “Method for Analysis of Abnormal Body Tremors”, U.S. Pat. No. 10,064,582 titled “Noninvasive Determination of Cardiac Health and Other Functional States and Trends For Human Physiological Systems”, and JP 6,130,474 with an English translated title of “Weight scale device and pulse wave velocity acquisition method”. The disclosures of all these patents and applications are incorporated by reference in their entireties.

### SUMMARY

In a first aspect, the disclosure provides a system for detecting symptomatic tremors. The system includes a seat sensor to measure at least a portion of weight from the user while using a seat and a foot sensor to measure at least a portion of foot weight from the user while the user is using the seat and supporting a foot with a foot support. The weight data from the seat sensor and the foot sensor is used to identify a temporary changes in the weight data which correlate with the manifestation of symptomatic tremors. An additional, or tertiary, force sensor may also be used with the system to provide additional data relevant to the detection of symptomatic tremors.

In a second aspect, the disclosure provides a method for detecting a potential symptomatic tremor in a user. The method includes providing a seat sensor and a foot sensor, monitoring the weight measured by those sensors, analyzing the weight data, and using the results of the analysis to generate a report related to the user's health. The seat sensor measures at least a portion of seat weight from the user while sitting on a seat. The foot sensor measures at least a portion of foot weight from the user while the user is sitting on the seat and resting a foot on a foot support. At least one part of the analysis of the data is to identify temporary changes in the weight data that correlates with the manifestation of symptomatic tremors.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is an isometric view of a toilet according to one embodiment according to the present disclosure.

FIG. 2 is a top view of the toilet of FIG. 1.

FIG. 3 is a view of the bottom of the seat and lid of the toilet of FIG. 1.

FIG. 4 is a view from the side of the toilet of FIG. 1 with the cover removed.

FIG. 5 is a view showing the top of a foot scale used in one embodiment of the invention.

FIG. 6 is a view of the bottom of the foot scale of FIG. 5.

FIG. 7 is an isometric view of a toilet used in another embodiment according to the present disclosure.

FIG. 8 is a top view of the toilet of FIG. 7.

FIG. 9 is a view of the bottom of the seat of the toilet of FIG. 7.

FIG. 10 is a partial view of the toilet of FIG. 7 with the cover removed.

FIG. 11 is a detail view of a handle used in an embodiment according to the present disclosure.

FIG. 12 is an isometric view of a mobile chair according to one embodiment according to the present disclosure.

FIG. 13 is an isometric view of a stationary chair according to one embodiment according to the present disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, xray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, “neural network”, “neural net”, and similar terms are meant to refer to a set of algorithms, modeled loosely after the human brain, that are designed to recognize patterns. They interpret sensory data through a kind of machine perception, labeling or clustering raw input. The patterns they recognize are numerical, contained in vectors, into which all real-world data, be it images, sound, text or time series, must be translated.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, the term “microfluidics” is meant to refer to the manipulation of fluids that are contained to small scale, typically sub-millimeter channels. The prefix “micro” used with this term and others in describing this invention is not intended to set a maximum or a minimum size for the channels or volumes.

As used herein, the prefix “nano” is meant to refer to something in size such that units are often converted to the nano-scale for ease before a value is provided. For example, the dimensions of a molecule may be given in nanometers rather than in meters.

As used herein, “bind” and similar variants are meant to refer to the property of facilitating molecular interaction with a molecule, such as interaction with a molecular biomarker.

As used herein, “tremor”, “body tremor”, and similar variants are meant to refer to involuntary motion, particularly those related to repeated muscle contraction and relaxation leading to shaking movements in one or more parts of the body. This shaking may be considered rhythmic or cyclical because of the fairly consistent and repetitive motion of the tremor.

As used herein, “physiologic tremor” and its derivatives are meant to refer to a fine tremor resulting from normal body function such as heartbeat, maintaining a posture, and movement. Occurrence of these is normal and generally not cause for seeking additional health or wellness care.

As used herein, “exacerbated physiologic tremor”, “noticeable physiologic tremor”, and their variants are meant to refer to a physiologic tremor which has become more pronounced than normal and is generally an indication of factors which may warrant health or wellness consideration or care, such as extreme fatigue, stress, intense emotion, low blood sugar (hypoglycemia), an overactive thyroid, medications such as corticosteroids, amphetamines or beta-agonists, heavy metal toxicity, stimulants such as caffeine, fever, and alcohol withdrawal.

As used herein, “symptomatic tremor”, “abnormal tremor”, “atypical tremor”, and their variants are meant to refer to exacerbated physiologic tremors. They also include tremors whose existence is symptomatic, suggestive of, or correlates with an abnormal or atypical health or wellness condition, which condition may warrant additional care or consideration. It includes tremors that correlate with abnormal conditions and circumstances such as multiple sclerosis, stroke, traumatic brain injury, neurodegenerative diseases that affect parts of the brain (such as Parkinson's disease), use of certain medicines (including particular asthma medication, amphetamines, caffeine, corticosteroids, and some drugs used for psychiatric and neurological disorders), alcohol abuse or withdrawal, mercury poisoning, an overactive thyroid, liver or kidney failure, and anxiety or panic.

In general, “weight” refers to the force excreted by a physical object or organism, especially a person or animal, under the influence of a gravitational field. As used herein, “weight” is sometimes used to represent the more general term “force”, which represents the mass of the physical object or organism multiplied by the acceleration of that mass. On the surface of Earth, gravity applies a relatively constant acceleration to mass thereon, thus creating the force of weight people are generally familiar with. When measured, the weight or force itself is generally not directly measured, but reaction forces acting in opposition to the weight or force are being measured.

The force exerted by a tremor is created by motion of one part of a person's body relative to another part of the body. While gravity does not control amount of force, it may influence the final measurement of the force. Thus, the force exerted by a tremor may be measured in a gravity environment different from that of a person on the surface of the earth so long as the effects of environment's gravity are accounted for by the system. This includes environments termed “weightless” wherein the person or environment is in a state of falling relative to large gravity objects in the vicinity (such as being in a diving aircraft or in orbit around a planet), resulting in the sensation of being free of gravity. As such, the force being measured to detect a tremor from a person is more dependent on the person's movements than from the gravitational pull of Earth; this force may manifest as a temporary change to measured weight. Thus accelerometers, while not the simplest way to measure the weight of a person at rest on Earth, are an acceptable form of weight sensor to detect the cyclical loading and unloading of forces associated with tremors. In short, this disclosure is not meant to limit the invention to applications at rest on the surface of a planet or other environment with similar gravity.

As used herein, “seat” and similar terms are meant to refer to a structure designed to receive force exerted from the rear of the legs and proximal portions of the body.

As used herein, “backrest” and similar terms are meant to refer to a structure designed to receive force from a person's back.

As used herein, “foot support” and similar terms are meant to refer to a structure designed to receive force from a person's foot, feet, and/or lower leg(s). This includes a structure that limits a single degree of freedom, such as one that rests on a floor and feet are placed thereon, as well as a structure meant to limit multiple degrees of freedom, such as a foot, ankle, or lower leg restraint.

As used herein, “mobile chair” is meant to refer to a propellable chair, such as a wheelchair or motorized scooter. They are generally capable of transporting a user through manually pushing, manually turning a wheel in contact with the ground, or a small motor. They generally contact the ground or floor via wheels or some other mechanism for providing motion to the chair (such as a tread spanning at least 2 wheels or gear like employed by some tanks and construction equipment, feet that can reposition themselves to provide a walking motion, or an air pocket like that employed by hover craft).

### Exemplary Embodiments

The present disclosure relates to a system for detecting body tremors, particularly those symptomatic of an abnormal health or wellness condition for which additional health or wellness care may be desired. The system includes at least two sensors for detecting the weight and/or force exerted by an individual: one which measures weight and/or force on a seat and one which measures weight and/or force on a foot support. The seat sensor is positioned such that it can measure forces a person may exert while seated (e.g. from the rear of the upper legs and adjacent portions of the body). The foot sensor is positioned so it can measure forces from a person's feet and/or lower legs while the person is seated on the seat. The shifting of force, especially weight, back and forth between the seat sensor and the foot sensor can be analyzed to identify a potential tremor. The data with the potential tremor can then be used for a variety of purposes, including comparing the potential tremor data to historical, relevant data; prompting the user to seek additional health or wellness care; reporting the data to a health, wellness, or other care provider; and logging the data for use in future comparisons. Additional sensors can be used to cooperatively detect seat force, cooperatively detect foot force, or additionally detect force applied elsewhere (herein generally referred to with the modifier “tertiary”), such as a backrest, handle, or other structure supporting weight or force, including force not being detected by the seat or foot sensors.

One benefit of the present disclosure is that it allows for identification of potential tremors and can even provide information that can be used by a trained person to help diagnose a tremor as a tremor. It also allows a trained person to monitor tremor activity. A further benefit is that all of this can be implemented for use during a person's normal routine, such as a person's normal restroom routine, and thereby provide regular tracing of tremor or potential tremor activity without having to deviate significantly from their normal routine. The more convenient it is to use the system, the more likely the person is to use it and use it consistently. For example, if implemented into the toilet at a person's home or care facility, the person can simultaneously use the restroom and be monitored for tremors without having to travel to an outside location. Similarly, if implemented with a person's seat, chair, or wheelchair, monitoring can be performed during the person's normal routine. This also can facilitate more frequent monitoring, providing more data with which to assess a person's tremor health. As another example, if implemented at a doctor's office, airport, or laboratory, a person can be monitored for tremors through the familiar activity of sitting down, such as one might do when going to the restroom, waiting in a waiting area, or riding transportation from one location to another, rather than by using an unfamiliar apparatus which requires less familiar activities to complete the monitoring.

Using an unfamiliar system for the monitoring can induce stress in the user and/or increase the chance the user will misuse the apparatus, any of which can negatively affect the quality of the measurement results. Additionally, users may be less likely to use an unfamiliar apparatus, resulting in fewer or no measurements.

As described above, the general application is to use a seat sensor and a foot sensor to detect and measure a portion of force from a user, including weight. After which, the data from those sensors is used to identify potential tremors within the force or weight data, which may then be used for a variety of purposes. There are a variety of ways to implement each of these elements, the selection of which depends on many factors, some of which factors are outside the scope of the invention, such as designer preference, cost, laws, regulations, consumer and stakeholder preferences, and various other market conditions. For example, in one embodiment, the seat sensor is used in conjunction with a toilet seat; in another embodiment, it is used in conjunction with a wheelchair; and in yet another embodiment, the seat sensor is used in conjunction with a more generalized structure that supports force from the rear of the upper legs and/or from the feet (such as a chair on the floor or a restraint system in a space vehicle). Each of these embodiments provides a significant number of acceptable configurations capable of achieving the functional goal of detecting at least a portion of the seat force and generating data based on that force. Similarly, each provides a significant number of acceptable configurations for the implementation of the foot sensor as well as for the analysis and use of the sensor data. Thus, the embodiments disclosed below are a sampling of specific or preferred possible configurations of the elements of the invention and should not be interpreted to limit how the spirit and scope of the invention are achieved.

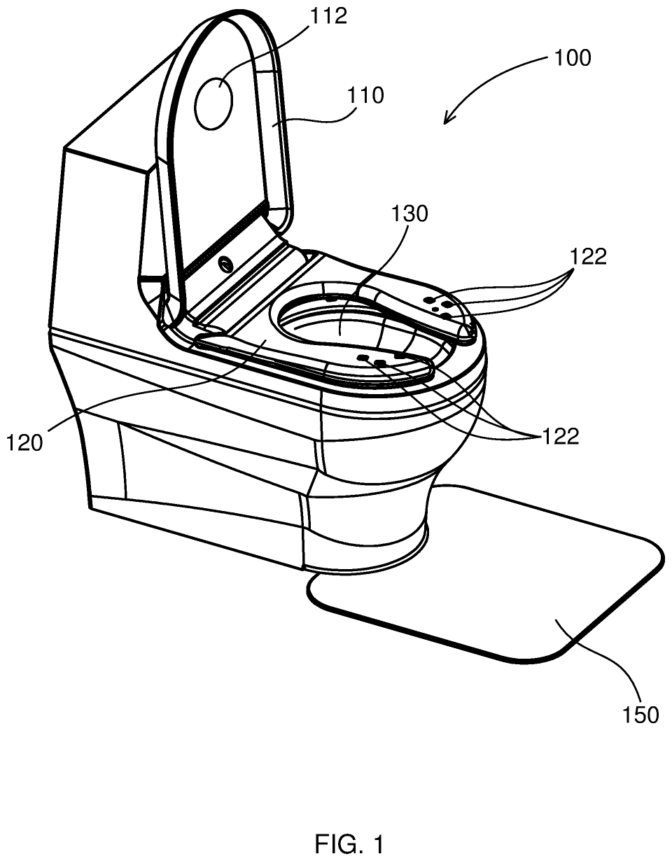

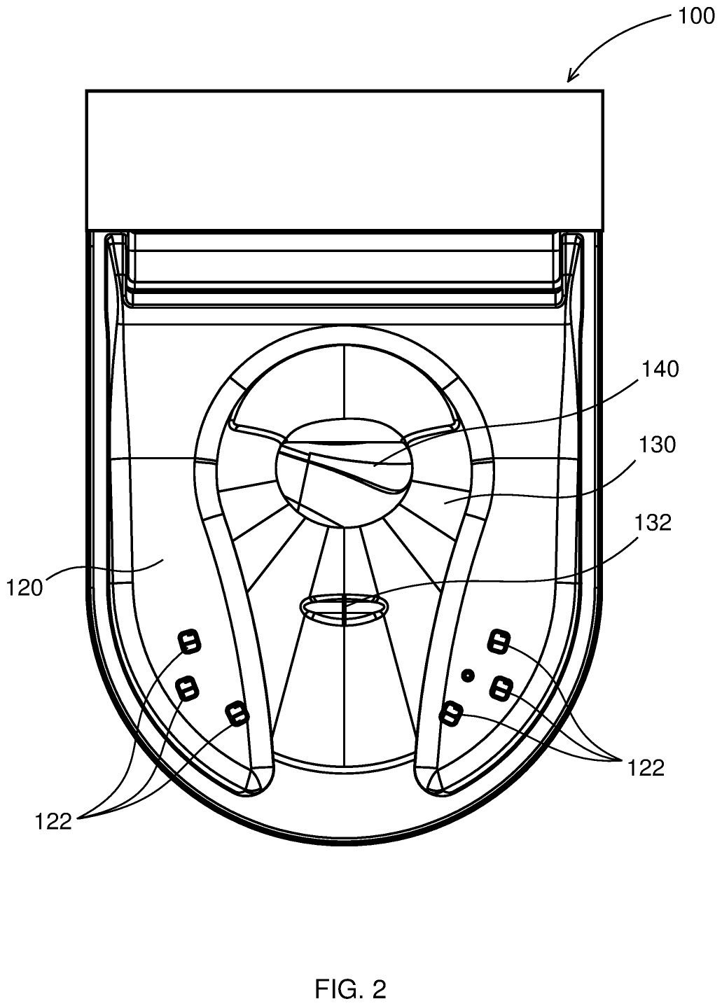

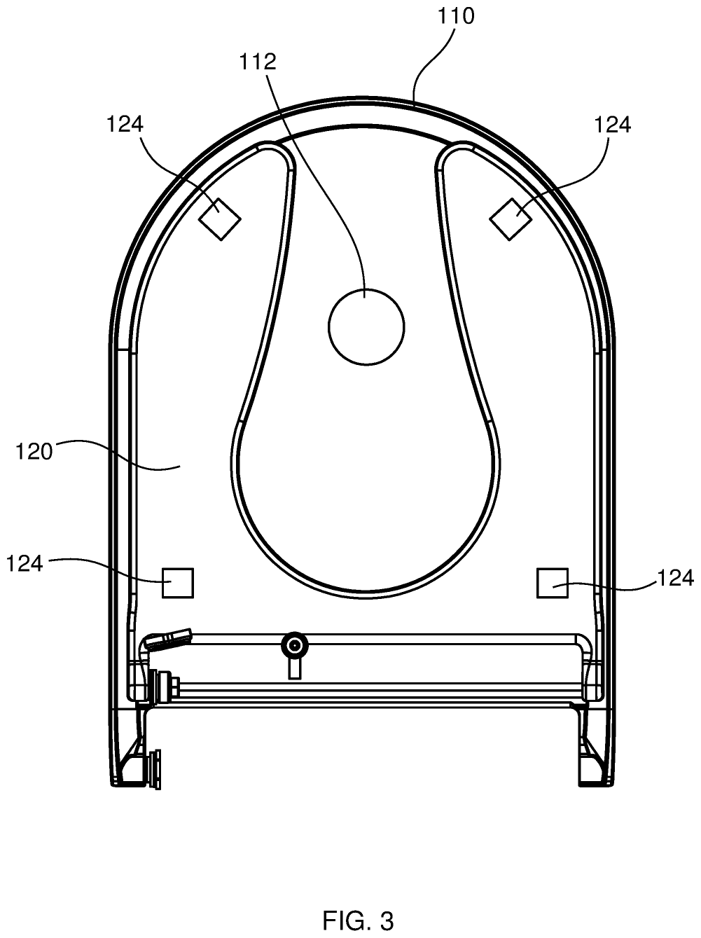

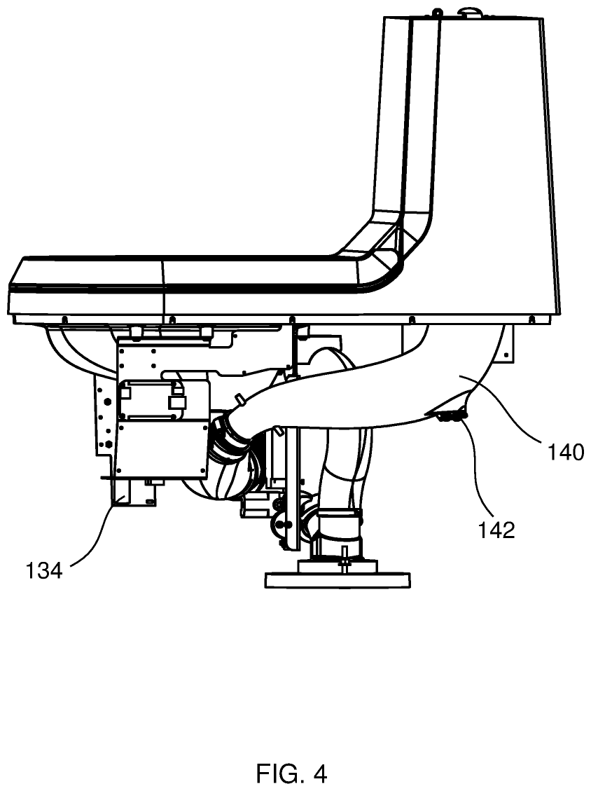

Now referring to FIGS. 1-4, one preferred embodiment of the toilet used in the system is shown. FIG. 1 shows an isometric view of toilet 100 with lid 110 open, showing seat 120 with multiple PPG sensors 122, bowl 130, and foot scale 150. FIG. 2 shows a top view of toilet 100 with lid 110 open, showing seat 120 with multiple PPG sensors 122, bowl 130, and excreta volume measure tube 140. Bowl 130 includes urine slit 132, which captures urine for reading by spectrometer 134. In one embodiment, seat 120 is hingeably attached to toilet 100 in a manner to decrease or minimize the amount of weight transferred from the seat to the hinge and increase or maximize the amount of weight transferred from the seat to toilet 100 away from the hinge; this can facilitate detection and measurement of seat weight by seat force sensors. FIG. 3 is a detail view of the underside of seat 120 with lid 110 behind seat 120. On the underside of seat 120 are seat sensors 124. Seat sensors 124 are positioned so they transfer force from seat 120 to the surface of toilet 100 directly below seat 120 when it is in the lowered position. Shown on lid 110 is stethoscope 112, which includes a microphone for recording audio sounds from a user's trunk portion of the body, Alternatively, lid 110 has a force sensor, such as an accelerometer, to detect and/or measure the force applied from the user leaning against lid 110, such as when seated on seat 120. FIG. 4 is a detail view showing some of the internal components of toilet 100, including urine volume measure tube 140, urine tube volume sensor 142, and other hardware 134, which can include a processors for receiving data from the sensors. Alternatively, the processors may be located in the water tank. Alternatively, the processor is remote and the signal from the sensors is transmitted (wired or wirelessly) to the processor.

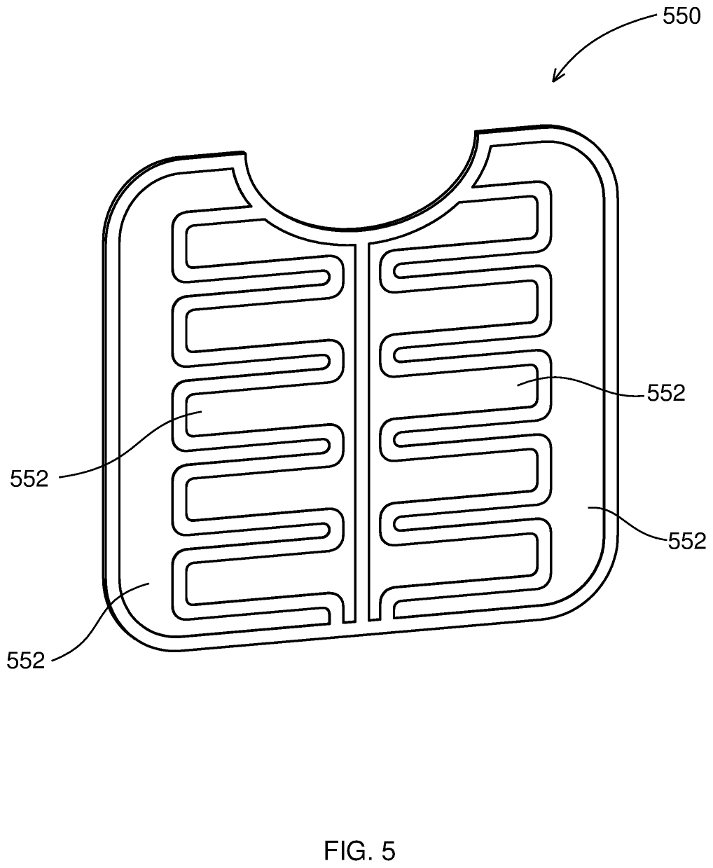

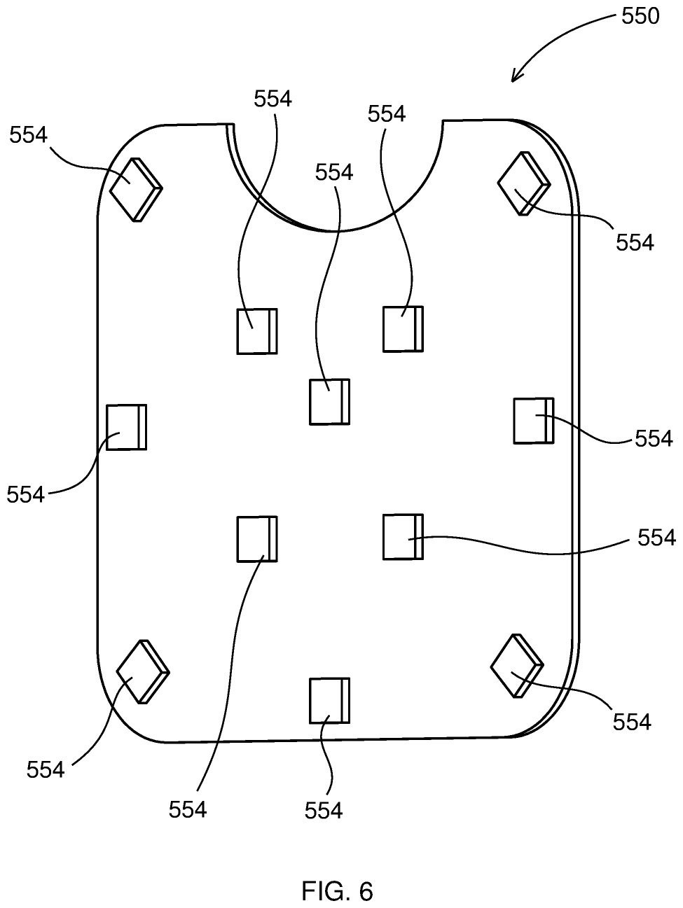

FIGS. 5-6 show one embodiment of a foot pad or foot scale that could accompany a toilet, especially one like that shown in FIGS. 1-4. FIG. 5 shows the top surface of foot scale 550 with bioimpedance sensors 552. To use the bioimpedance sensors, a user would place their bare feet on the top surface, contacting two bioimpedance sensors, allowing the user's bioimpedance to be measured. FIG. 6 shows the bottom of foot scale 550 with multiple foot sensors 554 disposed on the bottom surface such that they can transfer force from foot scale 550 to the surface(s) supporting foot sensors 554 (e.g. a floor). One side of foot sensor 554 attaches to the bottom of the scale and the opposing side of sensor 554 contacts the ground. Thus, when a user exerts weight on foot scale 550, the various foot sensors 554 can cooperate to determine the weight and/or force the user is exerting on the scale. More preferably, they cooperate to measure changes to the force the user is placing on the scale. Still more preferably, the focus of the measured changes to the force is to identify those which correlate with symptomatic tremors.

In one preferred embodiment, a user walks onto scale 150, and sits down on seat 120, leaving their feet on scale 150. While the user is using the toilet, PPG sensors 122 monitor the user's upper legs; seat sensors 124 monitor the portion of the user's weight on seat 120—including minor, apparent fluctuations that are a result of user tremors; foot sensors 154 monitor the portion of the user's weight on foot scale 150, and bioimpedance sensors 152 determine the user's bioimpedance. More preferably, the seat sensors 124 monitor fluctuations in the person's weight in an order of magnitude and range that correlates with symptomatic tremors.

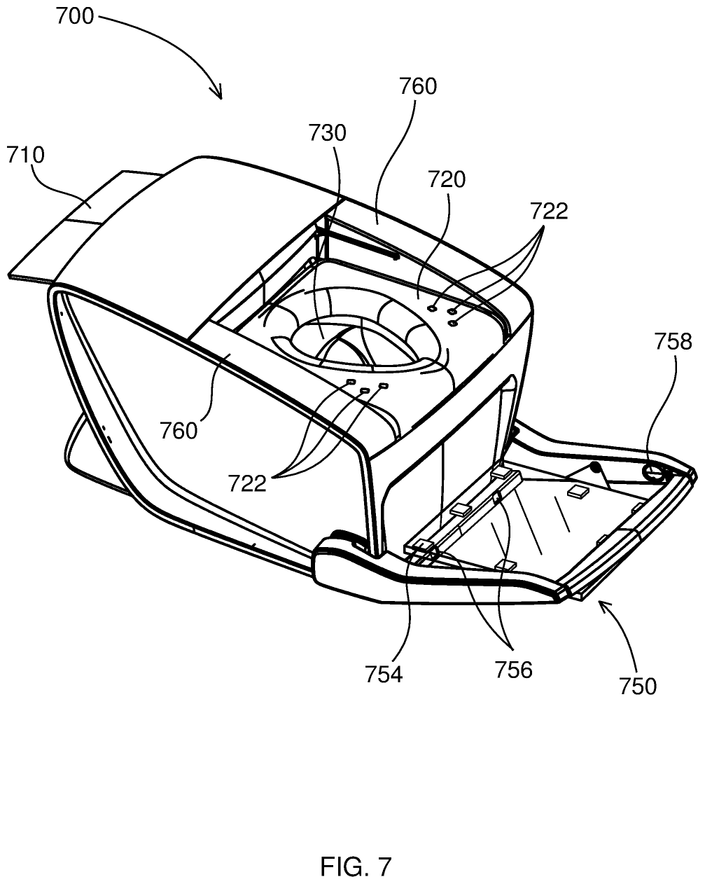

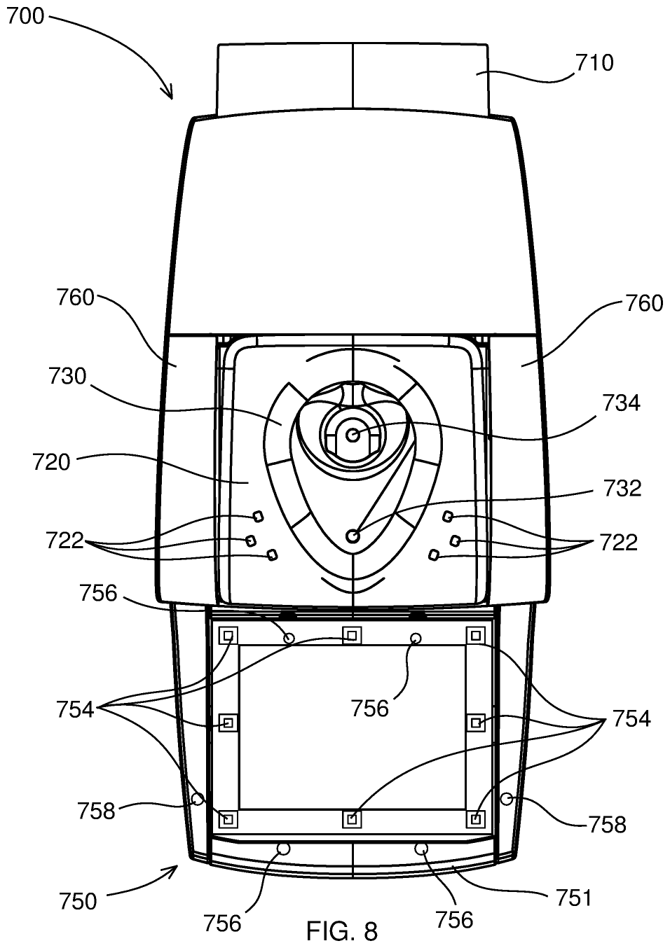

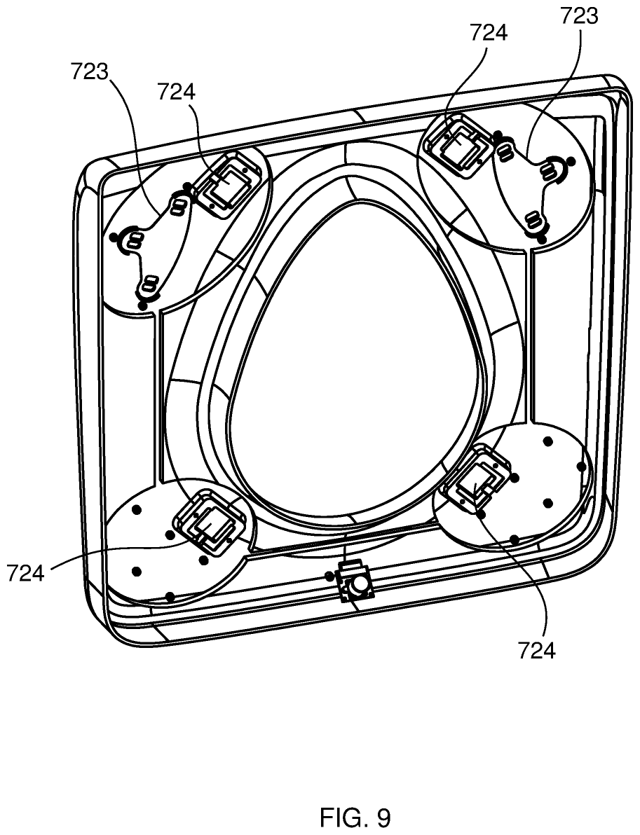

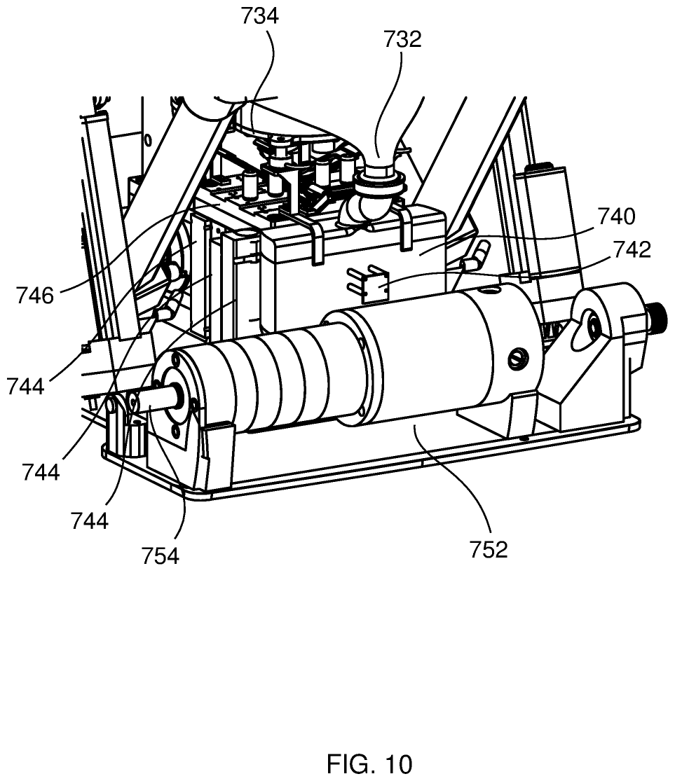

FIGS. 7-10 show another embodiment of the toilet. FIG. 7 shows an isometric view of toilet 700 with lid 710 open, showing seat 720 with multiple PPG sensors 722, bowl 730, foot platform 750, and handles 760. FIG. 8 shows a top view of toilet 700 with lid 710 open, showing seat 720 with multiple PPG sensors 722, bowl 730, foot platform 750, and handles 760. Bowl 730 includes urine receptacle 732 and fecal depository 734. In one preferred embodiment, handles 760 are in a recessed position and can be raised up relative to the toilet. FIG. 9 is a detail view of the underside of seat 720 showing seat sensors 724 on the bottom surface of seat 720. FIG. 10 is a detail view showing some of the internal components of toilet 700, including urine receptacle 732, fecal depository 734, urine volume measure chamber 740, urine spectrometer 742, science centers 744, fluid chip receptacle 746, foot platform motor and sensor 752, foot platform motor shaft 753. Foot platform 750 includes frame 751, a glass plate resting on multiple foot sensors 754, foot image sensors 756, and foot IR sensors 758. In one preferred embodiment, science centers 744 and fluid chip receptacle 746 are used in conjunction with excreta analysis, including urine samples and emulsified or otherwise processed excreta.

In one preferred embodiment, a user steps onto platform 750, sits down on seat 720, and platform 750 raises via motor 752 so the user's feet easily stay on the glass plate. While the user is using the toilet, PPG sensors monitor the user's upper legs; seat sensors 724 monitor the portion of the user's weight on seat 720—including minor, apparent fluctuations that are a result of user tremors; foot sensors 754 monitor the portion of the user's weight on foot platform 750, and sensors 754 and 758 monitor the user's feet and lower legs. More preferably, data from the force sensors are used to monitor for fluctuations in the force of an order of magnitude and range that correlate with symptomatic tremors.

In one preferred embodiment, sensors 754 and 758 are able to detect properties of the foot, including foot size and shape, coloring, and subdermal vascular properties. These images can undergo image recognition analysis, the results of which can be compared to preexisting data on the same to generate a report on a user's health. Additionally, data from other sensors can be turned into an image for analysis and report generation. Preferably, the report includes information relative to a user's vascular health. Preferably, the comparison is performed by a neural net which has been trained to recognize commonalities to and differences from preexisting images. When the preexisting images are coupled with known health states and/or conditions of the person from whom the images came, the neural net can suggest correlations between the user's images and health states and/or conditions (including neutral or positive ones). Additionally, when the neural net has examined previous data from the same user, the neural net can compare the user's prior state to his or her current state to report on the relative change. Therefore, it may be useful for user data to be averaged, have the mean taken, used in creating trend data, or otherwise be used in creating a baseline against which to compare new user data as it is generated.

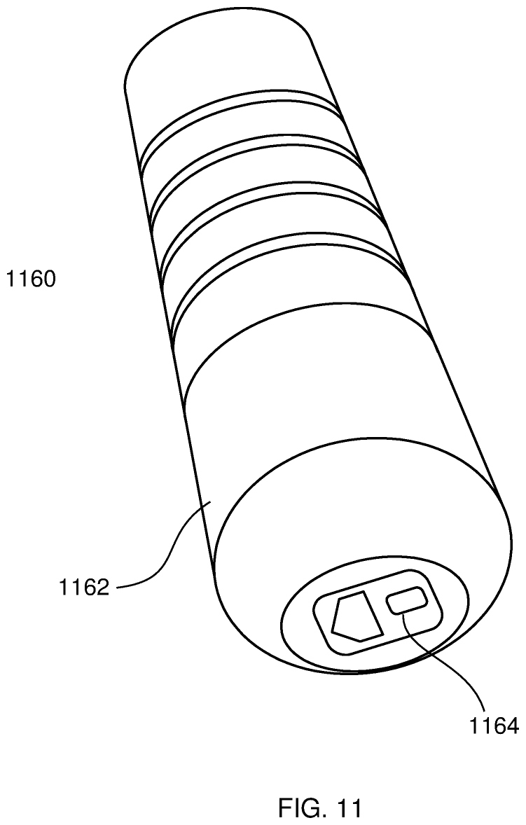

FIG. 11 shows an embodiment of a handle that could accompany a toilet or other apparatus a person may want hand or arm support while using. Handle 1160 includes electrical lead 1162 and PPG sensor 1164. Electrical lead 1162 could be a lead for a bioimpedance sensor and/or an ECG sensor. In one preferred embodiment, a handle would be connected to a cord (with wiring) that connects to the toilet. And another preferred embodiment a handle would be mounted to a structure adjacent to the toilet bowl. In either embodiment, a second handle could also be used. Additionally, a handle sensor could detect force from a person, including if the person uses the handle for support while sitting or transitioning between sitting and standing. A second handle may originate from the same connection point to the toilet or a location symmetrically opposite or mirrored from the first handle.

In one preferred embodiment, the seat sensor and foot sensor are integrated into a mobile chair, such as a wheelchair or motorized scooter. Such mobile chairs may be as simple as a common wheelchair with 4 wheels in contact with the ground, a seat for the user to sit on, and a footrest or foot restraint to hold the user's feet. Often, there will be one or two small wheels supporting the front portion of the chair and 2 large wheels supporting the rear portion of the chair. There is generally a structure or mechanism for propelling the chair, such as one or two handles behind the occupant which someone may use to push or pull the chair, a handle connected to a large wheel on each side of the chair so the occupant can propel themselves, and/or a motor which makes at least one wheel turn. There may be an additional steering mechanism, such as a handlebar which turns at least one wheel on an axis substantially perpendicular to the ground, a joystick, or a mouth-operated breathing tube. Mobile chairs also typically have a manually or electronically controlled wheel brake, axle brake, or other mechanism to prevent the chair from moving. Additional features on a mobile chair can include a backrest, an armrest, a headrest, a lap restraint to secure the occupant to the chair's seat, a foot restraint to secure the occupant's foot to a footrest, and/or making the footrest removable or repositionable. There may also be electronics, which could include a power supply, a controller or processor (which can record input from sensors, interpret user input, and/or generate output), a control accelerometer, a transmitter, and a receiver. While less common, a mobile chair may use a mechanism other than wheels for providing movement relative to the ground. One example of this is one or two treads which, similar to a wheel, roll relative to the ground to provide motion to the mobile chair.

Another embodiment is a mobile chair that makes use a pressurized layer of air between the ground and the chair. The chair sits on the air layer with enough clearance that it can move freely relative to the ground when propelled by another force; one common term for vehicles that float on pressurized air is “hovercraft”. The chair may create the pressurized air or the air may come from another source, such as a porous surface that receives the pressurized air from underneath (similar to an air hockey table). A variation of this makes use of magnetic or similar levitation rather than air pressure. Yet another embodiment depends on feet from the chair being in contact with the ground and the feet having relative motion to the chair to create a walking effect. A mobile chair may make use of more than one mechanism or method of propulsion.

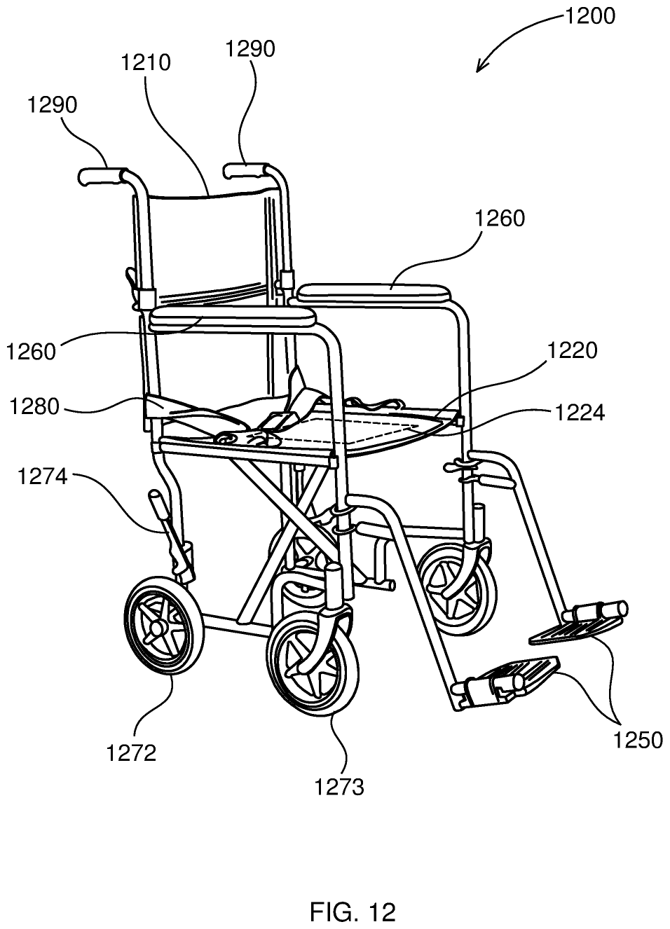

FIG. 12 depicts wheelchair 1200, which is one preferred embodiment of a mobile chair. It shows seat 1220, footrests 1250, wheels 1272 and 1273, brake 1274, armrests 1260, lap belt 1280, backrest 1210, and handles 1290. Lap belt 1280 may be used to secure an occupant of wheelchair 1200 in or to the same. There are a variety of ways to incorporate one or more seat sensor, foot sensor, and/or tertiary sensor to measure weight and other forces. In one preferred embodiment of the invention, seat sensor 1224 is internal to seat 1220. In another, a seat sensor is in an apparatus placed on or attached to the top of the seat. In yet another embodiment, a seat sensor attaches the seat to the frame of the wheelchair, transferring force from the seat to the rest of the wheelchair. In one preferred embodiment, the foot sensor is integrated into the substantially horizontal portion of footrest 1250. In another embodiment, the foot sensor is positioned on or in contact with the bar that connects the substantially horizontal portion to the frame of the wheelchair. In yet another embodiment, the footrest sensor is incorporated into an apparatus which slips on or attaches to the substantially horizontal portion of footrest 1250. Additionally, there may be a tertiary sensor used with each armrest to measure forces on each. The sensors may be connected to a portable power supply (such as a battery). They may transmit their respective signals to a processor, receiver, or other device on the wheelchair or within range of the wheelchair (not shown).

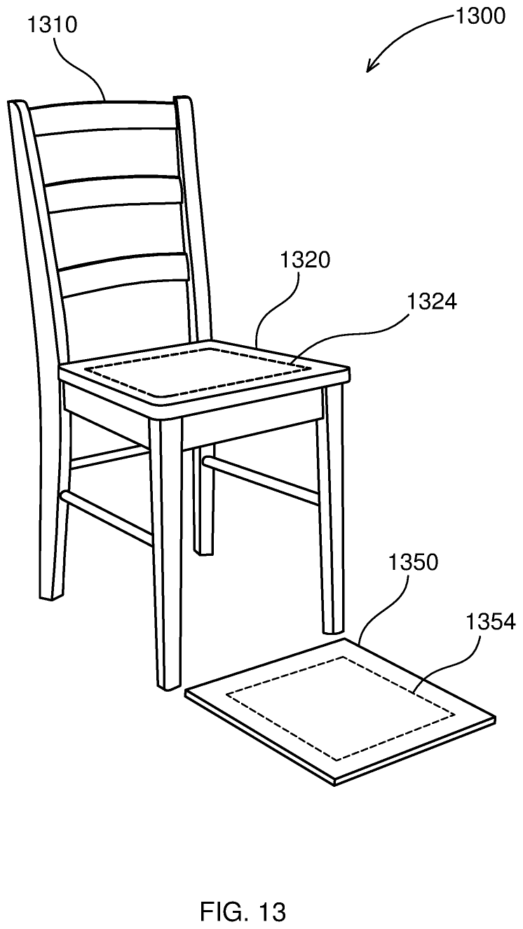

In one preferred embodiment, the seat sensor and foot sensor are used with a stationary chair. The seat sensor measures force exerted on the seat, including weight placed thereon. Similar to the toilet embodiments above, the foot sensor could be in a device which rests on the floor or in a device which is supported by the chair. FIG. 13 depicts chair 1300, which is one preferred embodiment of a stationary chair. It shows seat 1320, floor pad 1350, and backrest 1310. There are a variety of ways to incorporate one or more seat sensor, foot sensor, and/or tertiary sensor. In one preferred embodiment of the invention, seat sensor 1324 is internal to seat 1320. In another, the seat sensor is part of an apparatus placed on or attached to the top of the seat. In yet another embodiment, one or more seat sensors are between the seat and the frame of the chair, transferring the force from the seat to the rest of the chair. In one preferred embodiment, foot sensor 1354 is integrated into floor pad 1250. In another embodiment, a footrest attached to the chair replaces the floor pad. In an alternative embodiment, the chair has or is positioned next to an armrest or similar structure for supporting the hands and/or arms. In such an embodiment, a tertiary sensor could monitor the force received by the armrest. The force sensors may be connected to a portable power supply (such as a battery). They may transmit their respective signals to a processor, receiver, or other device on the chair or within range of the chair (not shown).

In one preferred embodiment, the seat sensor and foot sensor are integrated into a recliner, a chair which has motion relative to itself to allow the occupant to switch between a sitting posture and a more inclined posture. A common feature of many recliners is a foot support in the form of a footrest which angles horizontally outward from the area below the seat and which lifts the feet and legs by supporting the lower legs. A backrest generally also adjusts in angle, so the upper portion of the backrest moves horizontally away from the seat. The result is that the chair supports the person in a semi-inclined or reclined position; some recliners transition so the occupant is essentially lying down. There are numerous examples where a recliner is part of a couch that is wide enough to seat multiple people. There are versions that are manually operated, such as a lever whose angle sets the position of the foot rest or a lever which releases the footrest so it may extend; in the latter case, the footrest may be reset for the sitting position by using the foot and/or lower leg to press the footrest back into its locked position. There are also versions of recliner that are electronically operated with at least one motor to change the angle of the footrest and/or backrest. In a recliner embodiment, the seat sensor can measure force from the rear of the upper portion of the legs and the foot sensor can measure force from the lower legs. The sensors may be connected to a portable power supply (such as a battery). They may transmit their respective signals to a processor, receiver, or other device on the wheelchair or within range of the wheelchair (not shown).

Alternatively, there are some chairs and recliners where the seat portion of the chair can angle and/or reposition relative to the floor; one purpose of these chairs and recliners is to assist people in getting into and/or out of the chair. Not all chairs whose seat can change angle relative to the floor have a reclining footrest. Additionally, where the seat can change angle, the seat sensor and/or the foot sensor may also be used to monitor the user's weight distribution to assist with user safety and comfort while raising or lowering of the seat.

The use of the invention is not limited to environments where the only external forces are gravity or even to environments where the interaction with gravity is that of being on the surface of the planet. For example, one embodiment of the invention may be placed on or in an environment that experiences external forces unrelated to gravity, such as a tall building that sways in the wind, a floor prone to oscillations, a boat subject to waves, a location affected by earthquakes, a vehicle driving on the road, or an airplane in flight. Another embodiment of the invention might be used in orbit where the effects of gravity on a person feel negated because the person and the vessel carrying the person are both falling relative to Earth with approximately the same speed.

In one preferred embodiment, the seat and foot support each have a strap or other securing mechanism so the sensor can experience forces pushing on and pulling away from the seat and foot support.

Preferably, there is a common structure connecting the seat and foot support which, in the absence of a tremor, allow little to no motion of the seat and foot support to each other. There are many examples of this throughout the application. An example in addition to those mentioned is that of a brace attached at or near the waist and at or near the feet or lower leg. The “seat” which the seat force sensor is attached to may be the portion of the brace that contacts the upper legs or lower back (including a restraint). The “foot support” which the foot force sensor is attached to may be the portion of the brace that contacts the feet, ankle, or lower legs (including a restraint). Alternatively, a separate seat force sensor and foot support sensor may not be necessary. For example, a strain gauge attached to the brace between the seat and foot support could be used to detect reactionary forces in the brace due to a tremor.

In another embodiment, an accelerometer or other force sensing device may be used to monitor the general forces acting on the vessel or environment in which the seat and foot support are in. This sensor can be used as a control signal to remove external forces from the seat sensor data, foot sensor data, and any tertiary sensor data. In such an environment, such a control signal may be critical in differentiating between force changes that result from a tremor and force changes resulting from environmental forces.

There are many ways to detect the motion or forces that correlate with tremors, particularly symptomatic tremors which generally manifest with greater amplitude than physiological tremors. There are many types of force sensor that detect and/or measure a property of physical motion. For example, some load cells operate by deflecting a bar and a strain gauge attached to the bar generates a signal based on the amount of deflection. Similarly, some accelerometers also create a signal based on deflection. Such accelerometers can be constructed with a mass physically in contact with a piezoelectric crystal or other electric device. As the accelerometer moves, the mass mechanically strains the crystal or device and the crystal or device outputs a signal that correlates with the strain. Another type of force sensor converts the force placed on a sensor to pressure and outputs a signal based on the pressure.

When a series of instantaneous force or displacement signals are combined, an understanding of the tremor forces, waves propagation, and body motion for that period of time forms that help correlate the data with the various kinds of tremors. For instance, characteristics such as force, position, velocity, and/or acceleration can be used to determine the strength and frequency of the tremor, both of which are tools for classifying tremors.

A single weight or force sensor which measures a portion of the weight or force a person exerts can be used to monitor increases or decreases in the measurement. As described above, tremors or potential tremors can be gleaned from the data. For example, heartbeat can create a physiological tremor detectible by a cyclical changing, or repeated rising and falling, of weight. As another example, a symptomatic tremor may cause a person to rock forward and backward, which would similarly register as repeated rising and falling of the measured force, though generally on a much larger scale than that resulting from heartbeat.

A second sensor can independently do the same as a first sensor. But when the first and second sensor's data is used together, the combination of the data can be used to gain additional information, including whether weight is shifting back and forth between 2 sensors. This would manifest as a relatively constant total measured weight while the weight goes up on one sensor and down on the other. The shifting of weight between sensors can facilitate the determination of the center of force of the forces being detected (i.e. a theoretical point location with forces and moment applied which mimics the actual forces, resulting in the same measurement of the applied forces—similar to the center of gravity for a weight or center of mass for a mass) and whether force is being placed on additional structures the seat and weight sensors are not detecting (e.g. the floor, a handle, an arm rest, a back rest, the tank of a toilet, a counter, or a table). Of particular note, the system does not require detecting a person's entire weight to assess tremors.

For example, when a person is seated with weight being measured on a seat sensor and a foot sensor, a symptomatic tremor may cause the person to cyclically shift some of their weight to a handle or back rest. In this scenario, the combined weight measured by the seat and foot force sensors may change as weight shifts to and from an armrest. In one preferred embodiment, the system functions by measuring cyclical loading and unloading of approximately the same amount of the person's weight during a tremor or potential tremor, which may include a relatively constant average portion of weight (the change may accommodate events such as voiding bowels or changing the amount of weight applied to a non-weight-detecting structure).

A symptomatic tremor may cause a person to exert force in addition to their weight. If the tremor causes a person to cyclically push and/or pull on one structure, they may simultaneously be pushing and/or pulling on another structure. For example, if a person is seated with weight being measured on a seat force sensor and a foot force sensor, a tremor may cause them to lean back and push against a back rest. This may result in reactionary forces on the seat and/or foot support, which register on the respective seat and/or foot force sensors. These reactionary forces may increase the measured force to a value higher than the person's weight.

A symptomatic tremor may also induce vibration or waves that travel through the body. A single force or weight sensor can be used to detect and measure these tremor vibrations. Additionally, two sensors which detect change in weight or force from a user may be able to be used to provide origin location estimates for a tremor. Analysis of the data from each sensor can be used to determine how out of sync the vibrations are at the monitored location. The distance between the two locations can be used in conjunction with materials of the body to estimate a transit time of how long it would take for a vibration starting at one location to each the other location; these practices are known in fields concerned with determining location based on a measurable sign, including GPS triangulation and earthquake epicenter science. In one preferred embodiment, this transit time can be compared to the out of sync time difference of the measurement of the vibrations at each location. Based on the comparison, an estimate of the tremor location origin may be made. For example, if the out of sync difference is shorter than the transit time, the location may be estimated as between the two locations; and more likely estimated to be on an imaginary line or surface between the two locations. If the transit time is equal to the difference, it may be estimated that the tremor origin location is at or on the other side of the first location which receives the vibration relative to the second location; and more likely on an imaginary line or surface that passes through the two locations. If the transit time is less than the out of sync difference, it may be estimated that the tremor origin location is not on an imaginary line or surface that passes through the two locations. Other rules may also apply. A third such sensor can significantly increase the accuracy of origin location estimates. Each additional sensor can serve to refine the accuracy of origin location estimates.

There are many ways to handle the data and signals generated by the sensors and the data and signal from any single sensor can be handled differently from that of any of the others. The goal is to provide one of a multitude of functional outcomes with the sensor data. The list of possible outcomes includes gathering population data to create a database for future comparison related to health and wellness; and using the data to create a current or future report on the user's health and wellness. The report generated may simply notify the user of a successful generation of data; communicate whether the user may want to seek additional health or wellness care; give a more detailed assessment of the user's health and wellness state; or provide health and wellness information about the user to a health or wellness provider. The selection of how to handle the data will depend on many factors, including market conditions. For example, in one embodiment, the sensor data can be gathered to one or more processor in the toilet, processed locally, and a report displayed to the user. In addition to the displayed report, after local processing, data and/or its derivatives can be sent to a processor outside the toilet, such as the cloud or a mobile device. Alternatively, the data can be sent (wired or wirelessly) to an external processor for processing. The signal from any sensor can go to a processor as raw data or go through a signal processor (such as a digital signal processor (DSP)). Thus, the elements of the data management portion of the system can vary and still achieve the desired functional outcomes.

In one preferred embodiment, user data, including data related to tremors, is compared to a predetermined standard based on preexisting data to create a report on the user's health. More preferably, the report includes information on the user's health related to tremors. The predetermined standard can include a variety of different factors, including the user's health and wellness history, a population's health and wellness history, and user and population demographic information which may be related to health assessments, including age, weight, height, gender, race, and environmental factors and/or exposures. Within these categories, relevant information may include a variety of different tremor classification methods and their associated criteria; frequency of tremor occurrence; known strength, force, or amplitude of tremors; a determination of which portion(s) of the body are affected by tremors; location measurements are being taken; likely reaction time for tremor motion; likely transit rate through the body for vibrations resulting from tremors; a person's personal and/or family history with tremors and related conditions. These factors may be used to create a general or individual baseline for what is healthy. It may be used to create different categories of health into which a user can be placed based on their data. The selection of which factors to use depend on many factors, including some outside the scope of the invention such as market conditions and stakeholder preference.

One way of comparing this data is to take cyclical data such as BCG, PPG, ECG data, or potential tremor data; perform waveform image recognition analysis on the data; and run the resulting image(s) through a neural net. Preferably, the neural net will have been previously trained through the input of preexisting data in the form of relevant images paired with known conditions associated with the person who the waveform was generated from represented by the preexisting data (including neutral or healthy conditions). The neural network may then associate features of the images with conditions and look for the same features in an image to be analyzed. Data and relevant images include those such as BCG, PPG, or ECG waveforms; graphs of potential tremor data; urine flow results; urine spectrographic results; durometer results; and stethoscopic results. Within the neural net, images of the user's data will be compared to the pre-existing data. The neural net will find similarities between the two sets of data which correlate the user's data to conditions the neural net has been trained to recognize and those correlations will be reported back. Alternatively, the data is uploaded to the neural net in a different format, such as an audio file or raw time-series data.

In another preferred embodiment, a variety of filters and rules are applied to the sensor data which result in the identification of tremor candidates in the data. More preferably, the tremor candidates are categorized into different types of tremors, including a non-tremor category for features identified as false positives. The data can be used to provide information such as the amplitude, frequency, length, force, speed, velocity, and acceleration associated with a tremor. There are many possible steps and rules which could be used in this assessment and as is discusses in more detail in other parts of the application, the determination of which ones to implement will depend on many factors outside the scope of the invention. In general, the signal can be processed to make it more usable, such as through the use of filters like low pass or high pass. Also generally speaking, rules can be applied to identify or classify potential tremors, physiological tremors, the various kinds of symptomatic tremors, and/or non-tremors.

U.S. patent application Ser. No. 16/888,024 titled “Toilet Configured to Distinguish Excreta Type” includes a detailed discussion of time-series and event focused data analysis. It is included herein in its entirety. In summary, the application addresses how to identify characteristic of the data that correlate with the desired events. A similar approach can be taken in the present disclosure for tremor identification by implementing algorithms and rules that are relevant to tremor identification rather than fecal event identification.

The system may focus on providing a user assessment based on general population data. Additionally, the system may also associate current and historical data with a specific user, track changes to the tremor from one measurement session to another, and report on how a tremor is trending over a longer period of time than a single use of the system.

There are a wide variety of acceptable choices for the geometry, materials, manufacturing processes, and other elements of the design, manufacture, and implementation, including the electrical hardware and software. The selection of these various elements depends on a number of factors, including costs, supply chain, availability of labor and materials, design forces and safety factors, designer and stakeholder preference, local laws and regulations, and other market conditions. For example, there are a wide variety of potential materials that the various elements of the invention could be made from. Regarding those that take weight and other forces, many materials can be designed to bear the forces associated with a user's weight and potential tremors. Therefore, numerous other factors outside the scope of the invention play into the selected design, such as space, weight, production and installation costs, and maintenance requirements.

Additionally, the general principle of monitoring two force or weight sensors does not have to be limited to a force or weight seat sensor and a force or weight foot sensor. As noted in other portions of the disclosure, a tremor is the repeated and unintentional contraction of a muscle and results in relative motion of one part of the body to another part. Thus, alternative embodiments simply need to assess relative motion of one part of the body to another. This may include the use of two force or weight sensors as noted in many of the mentioned embodiments, but replaces at least one of the seat or foot force measurements with the force measurement from a different part of the body. For example, the system could measure the force or weight change at any two of the following: a person's head, neck, chest, torso, abdomen, finger, hand, lower arm, upper arm, waist, upper leg, lower leg, ankle, foot, and toe. An alternative method includes using one or more camera (or other image sensor) to track the motion of specific parts of the body. Another alternative, like that noted in a previous embodiment, includes the use of a single sensor that measures the force between any two parts of the body, such as with a strain gauge attached to a structure connecting two parts of the body.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A system for detecting a symptomatic tremor of a user comprising:
a seat sensor to measure at least a portion of seat weight from the user while using a seat;
a foot sensor to measure at least a portion of foot weight from the user while the user is using the seat and supporting a foot with a foot support; and
wherein weight data from the seat sensor and the foot sensor is used to identify a series of temporary weight changes in the weight data which correlate with the manifestation of symptomatic tremors.

2. The system of claim 1, wherein the seat comprises a seat of a toilet.

3. The system of claim 2, wherein the foot support is adjacent to the toilet.

4. The system of claim 2, wherein the foot support is attached to the toilet.

5. The system of claim 1, wherein the seat and foot support are part of a mobile chair.

6. The system of claim 1, wherein data related to the symptomatic tremor is analyzed in conjunction with a database and wherein results are provided in a report to the user or a healthcare provider.

7. The system of claim 6, wherein the report includes an assessment of the user's tremor data relative to a predetermined standard.

8. The system of claim 6, wherein the database contains historical tremor data from the user and the weight data is compared to the historical tremor data to assess changes in manifestation of symptomatic tremors.

9. The system of claim 1, wherein data related to the symptomatic tremor is added to a database used to track health and wellness data for the user.

10. The system of claim 1, wherein weight data from the seat sensor and the foot sensor is used to identify the shifting of weight back and forth between the seat sensor and the foot sensor.

11. The system of claim 1, further comprising a tertiary sensor to measure tertiary weight or force from a user on a tertiary support structure and wherein force data from the tertiary sensor is also used to identify a series of temporary weight changes in the weight data which correlate with the manifestation of symptomatic tremors.

12. The system of claim 1, wherein at least one of the seat sensor and the foot sensor comprise a load cell.

13. The system of claim 1, wherein at least one of the seat sensor and the foot sensor comprise an accelerometer.

14. The system of claim 11, wherein the tertiary support structure comprises a handle or armrest that supports weight or force received from a user's hand or arm.

15. The system of claim 1, further comprising an additional seat sensor that cooperates with the seat sensor to measure the at least a portion of weight from the user while using the seat.

16. A toilet for detecting a symptomatic tremor of a user comprising:
a seat sensor to measure at least a portion of seat weight from the user while sitting on a toilet seat;
a foot sensor to measure at least a portion of foot weight from the user while the user is sitting on the toilet seat and supporting a foot on a foot support in front of the toilet; and
wherein weight data from the seat sensor and the foot sensor is used to identify a series of temporary weight changes in the weight data which correlate with the manifestation of symptomatic tremors.

17. A method for detecting a potential symptomatic tremor in a user comprising:
providing a seat sensor to measure at least a portion of seat weight from the user while sitting on a seat;
providing a foot sensor to measure at least a portion of foot weight from the user while the user is sitting on the seat and resting a foot on a foot support;
monitoring the weight measured by the seat sensor and the foot sensor;
analyzing weight data from the monitoring to identify a series of temporary weight changes in the weight data which correlate with the manifestation of symptomatic tremors; and
using the results of the analysis to generate a report related to the user's health and wellness.

18. The method of claim 17, wherein the analysis comprises uploading the data derived from the weight data to a neural network trained to identify features of such data that correlate with a symptomatic tremor, the neural network identifying features in the data that correlate with the manifestation of symptomatic tremors, and the neural network returning an assessment of the user's health based on the results of its tremor identification process.

19. The method of claim 17, wherein the analysis comprises identifying a series of temporary weight changes in the weight data which correlate with the manifestation of symptomatic tremors.

20. The method of claim 19, wherein the temporary weight changes have a frequency between 3 Hz and 30 Hz.
